(19) United States
(12) Patent Application Publication
(10) Pub. No.: US 2006/0009411 A1
(43) Pub. Date: Jan. 12, 2006

(54) Targeting compounds to a cell nucleus

(76) Inventor: Magdolna G. Sebestyen, Madison, WI (US)

Correspondence Address: MIRUS CORPORATION, 505 SOUTH ROSA RD, MADISON, WI, 53719, US

(21) Appl. No.: 11/220,214

(22) Filed: Sep. 6, 2005

Related U.S. Application Data
(63) Continuation-in-part of application No. 10/200,800, filed on Jul. 22, 2002.
(60) Provisional application No. 60/309,319, filed on Jul. 31, 2001.

(51) Int. Cl. A61K 48/00

(52) U.S. Cl. 514/44

(57) ABSTRACT

We disclose gene delivery systems that target exogenous nucleic acids to the nucleus of mammalian cells and are delivered to chromatin during cellular mitosis, remaining within the nucleus after mitosis.

### CROSS-REFERENCE TO RELATED APPLICATIONS

This application is a continuation-in-part of U.S. application Ser. No. 10/200,800, filed Jul. 22, 2002, pending, which claims the benefit of U.S. Provisional Application No. 60/309,319, filed Jul. 31, 2001.

### FIELD OF THE INVENTION

This invention relates to gene delivery systems which target exogenous nucleic acids to the nucleus of actively dividing mammalian cells during mitosis.

### BACKGROUND

All eukaryotic cells are divided into functionally distinct, membrane-bound compartments. The two major compartments pertinent to gene delivery are the cytoplasm and the nucleus. The two compartments are separated by the nuclear envelope (NE): two concentric membrane layers punctured by pores. The pores, called nuclear pore complexes (NPCs), are formed by supramolecular assemblies of multiple copies of some 30-50 different proteins (Pante N et al. 1996). NPCs allow the selective, active transport of macromolecules in both directions across the nuclear envelope provided they carry specific signals, or addresses, called nuclear localizing signals (NLS) and nuclear export signals (NES). These signals are recognized by receptor molecules, which in turn mediate translocation through the central channel of the pore (Nigg E 1997, Conti E et al. 2001). Macromolecules larger than 50-60 kDa do not efficiently cross the nuclear envelope without displaying such signals.

High transfection efficiencies, up to 100%, would be extremely beneficial for several research applications as well as for in vivo gene therapy. However, current methods to transfect genes into cultured cells with high efficiency often involve the use of viral vectors or are associated with high levels of toxicity. Viral gene delivery is likely to increase the chance of rejection in vivo after transfer due to display of viral antigens. High toxicity is associated with electroporation and high doses of cationic lipids. While these parameters may be acceptable for some in vitro applications, they are incompatible with many other in vitro applications and all in vivo gene therapy usage.

Most of the currently used non-viral gene delivery methods deposit the DNA into the cytoplasm. From there it must be transported to the nucleus in order for expression to occur. Thus, one of the major physical barriers for effective delivery of plasmid DNA (PDNA) into mammalian cells is the nuclear envelope. It is believed that the breakdown and reassembly of the nuclear envelope during mitosis allows entry of DNA into the nucleus and accounts for improved transfection efficiencies observed in dividing cells (Wilke M et al. 1996, Mortimer I et al. 1999, Brunner S et al. 2000). In fact, for some oncoretroviruses (e.g. MLV), whose preintegration complexes are unable to be transported through the NPC of the intact interphase nuclear envelope, nuclear entry depends on the breakdown of the nuclear membrane at the onset of mitosis. However, the disassembly of the NE alone is insufficient to ensure that the preintegration complex will partition to a newly formed nucleus at the end of mitosis. These viruses possess mechanisms to enhance retention of their genomes in the nucleus (Brunner S et al. 2000, Piolot T et al. 2001). Disassembly of the NE during mitosis results only in a very limited increase in expression of transfected genes. Studying the sub-cellular distribution of macromolecules after mitosis, we have shown that pDNA and large dextran are mostly excluded from re-forming nuclei. The molecular details of nuclear assembly at the end of mitosis suggest that only the chromosomes and molecules physically associated with them become enclosed within the new nucleus as the envelope forms closely around the chromatin (Ludtke J J et al. 1999). We postulate that this strict sorting mechanism is one of the reasons why marker gene expression efficiency remains far below 100%, even in actively dividing cultured cells.

Two conceptually different pathways can be used to accomplish the nuclear targeting of exogenous DNA in mitotic cells. First, the traditional nuclear localization signal (NLS) mediated process can theoretically promote the transport of pDNA molecules through NPCs. All published efforts for the enhancement of gene delivery to the nucleus have focused on this method. A variety of such NLS signals have been used in attempts to target exogenous DNA to interphase nucleus (Sebestyen M G et al. 1998, Ludtke J J et al. 1999, Subramanian A et al. 1999). We suspect that, like endogenous nuclear proteins, NLS-labeled DNA transported into the nucleus during interphase becomes excluded again from nuclei at the end of mitosis. The second method, proposed in this invention, describes associating a biologically active compound with mitotic components to increase the efficiency of nuclear uptake and retention of the biologically active compound in dividing cells.

### BRIEF DESCRIPTION OF FIGURES

FIG. 1. Sub-cellular location of 500 kDa dextran (lower left panel; A, B, C, D) and Cy5-labeled plasmid DNA (upper left panel; A, B, C, D) in undivided (A, C) or divided (B, D) cells 16-22 h after delivery into either the cytoplasm (A, B), or nucleus (C, D). The majority of cells divided by this time, but a small population of undivided cells remained. The EYFP-Nuc protein, encoded by the injected plasmid DNA, emits green fluorescence (upper right panel; B, C, D) and predominantly accumulates in nucleoli. In contrast, both the dextran and the DNA are excluded from the nucleoli of the undivided cell after nuclear delivery (C). In divided cells, both dextran and DNA are excluded from nuclei of the daughter cells independent of whether they were injected into the cytoplasm or the nucleus (D). The image in the bottom right corner of each panel is the merged image of all three channels. Images were collected using confocal microscopy. Each image represents a single 0.5 μm optical section.

FIG. 2. Distribution of the Ki-67 antigen at different stages of the cell cycle. Synchronized HeLa cells were probed with anti-Ki-67 MAb. Alexa488-anti-Mouse IgG (upper panel) and ToPro3 DNA staining (lower panel) are shown.

FIG. 3. Sub-cellular distribution of various Ki-67 domains expressed as EYFP-fusions in transiently transfected HeLa cells. EYFP-Ki fusion protein (upper panel; Interphase, Mitosis); ToPro3 DNA staining (lower panel; Interphase, Mitosis).

FIG. 4. Fluorescent micrograph showing targeting of Cy3-Streptavidin to mitotic chromosomes by an LNA-1 chromosomal targeting signal. A. Interphase cell. B&C. Mitotic cells. Upper left subpanel—Cy3-Streptavidin; Upper right subpanel—actin stained with Alexa-488-phalloidin; Lower left subpanel—DNA stained with TO-PRO®-3.

FIG. 5. Nuclear targeting of a 60 kDa protein by an anti-Ki-67 monoclonal antibody chromosomal targeting signal. A. Microinjected cell that underwent cell division. B. Undivided cell. Upper left subpanel—anti-Ki67 antibody detected with Cy3-labeled anti-IgG; Upper right subpanel—DNA stained with TO-PRO®-3; Lower left subpanel—Oregon Green-Streptavidin.

FIG. 6. Nuclear targeting of cargo compounds by a streptavidin-anti-Ki-67 antibody chromosomal targeting signal.
A. Fluorescein-Biotin cargo. Upper left subpanel—anti-Ki67 antibody detected by Cy3-labeled anti-IgG antibody; Upper right subpanel—Fluorescein-Biotin localization; Lower left subpanel—TO-PRO®-3 labeled DNA.
B. Biotin-BSA cargo. Upper left subpanel—Cy3-BSA localization; Upper right subpanel—anti-Ki67 antibody detected by Alexa Flour 488-labeled anti-IgG antibody; Lower left subpanel—TO-PRO®-3 labeled DNA.

FIG. 7. Targeting of 70 kDa dextran to the nucleus during mitosis by a anti-Ki-67 antibody chromosomal targeting signal complex during mitosis. The dextran was linked to the CTS via a non-covalent streptavidin-biotin linkage. A-C. Dextran-biotin-SA-antiKi67 IgG. D-F. Dextran-biotin-SA plus unconjugated antiKi67 IgG control. Upper left subpanels—Localization of Cy3-labeled 70 kDa dextran cargo. Upper right subpanels—Localization of anti-Ki67 IgG (detected by Alexa Fluor 488-labeled goat anti-Mouse IgG) Lower left subpanels—Nuclei stained with TO-PRO®-3 DNA dye.

### SUMMARY

In a preferred embodiment, we describe a process to increase targeting of a biologically active compound to the nucleus of a dividing cell as the cell proceeds through mitosis comprising associating the compound with a Chromosome Targeting Signal (CTS). This targeting signal is distinct from the traditional nuclear localization sequence (NLS), in that it does not require transport of the compound into interphase nuclei through nuclear pore complexes (NPCs). A CTS targets the cargo to which it is associated to the chromosomes during mitosis, resulting in enhanced localization within a re-assembled nuclei. The cargo can be a biologically active compound such as a protein, drug, or polynucleotide. The cargo can also be a molecule useful for biologically research, such as, but not limited to, a marker molecule. A CTS may be used to enhance nuclear localization of a compound in a cell that is in vivo or in vitro.

In a preferred embodiment, we describe a process for associating a biologically active compound with mitotic chromosomes resulting in partitioning of the compound to the nuclear compartment prior to the end of telophase. The CTS may be used to enhance nuclear localization of a compound in a cell that is in vivo or in vitro.

In a preferred embodiment, a CTS is used to prolong residence of a cargo molecule within the nucleus of a dividing cell. During reformation of the nuclear envelope at the end of mitosis, most compounds not associated with chromosomes are excluded from the newly formed nucleus. Without a functional NLS, these compounds do not gain re-entry into the nucleus. Association of a compound with a CTS increases its retention in the nucleus as the cell progresses through mitosis.

A preferred chromosome targeting signal is a Ki-67 protein, a functional protein fragment or peptide derived from the Ki-67 protein, or an antibody to the Ki-67 protein. Another preferred chromosome targeting signal is the latent nuclear antigen-1 (LNA-1) of human Herpesvirus 8, a functional protein fragment or peptide derived from the LNA-1 protein, or an antibody to the LNA-1 protein. Other proteins that associate with chromatin as the nuclear envelope is formed around the condensed chromosomes as the cell goes through mitosis may also function as chromosome targeting signals. To be a CTS, the protein must be incorporated into newly formed nuclei at the end of mitosis.

To facilitate nuclear localization of a cargo, the CTS must be linked to the cargo. The CTS may be associated with or attached to a molecule by a covalent linkage or by a non-covalent linkage. The linkage may or may not include a spacer group. A non-covalent linkage must be sufficiently stable to maintain the linkage of the cargo to the CTS in the cell as the cell progresses though mitosis. An example of a non-covalent linkage is a streptavidin-biotin or avidin-biotin non-covalent interaction, wherein the CTS is linked to a streptavidin or avidin and the cargo is linked to a biotin. Alternatively, the CTS can be linked to the biotin. The CTS is then associated with the biologically active compound through the well known streptavidin/avidin-biotin interaction.

In a preferred embodiment, a CTS may be used in combination with other functional groups or signals. These signals include, but are not limited to, cell targeting signals, nuclear localization signals, and membrane active compounds, and may aid in targeting the cargo to specific cells types, binding to cell receptors to aid in internalization, enhancing escape from membrane enclosed compartments such as endosomes or avoiding undesirable interactions such as with serum components.

In a preferred embodiment, a CTS can be used to deliver a toxic compound to an actively dividing cell such as a cancer cell. The toxic compound can be a nucleic acid that encodes a suicide gene.

Further objects, features, and advantages of the invention will be apparent from the following detailed description when taken in conjunction with the accompanying drawings.

### DETAILED DESCRIPTION

Several research groups have reported that mitosis enhances marker gene expression, believed to be aided by the breakdown of the nuclear envelope (NE) during cell division. However, even after delivery of large amounts of DNA directly into the cytoplasm, significantly less than 100% of cells express the injected gene following mitosis (Ludtke J J et al. 2002). When examining the localization of cytoplasmically microinjected fluorescent DNA in HeLa cells, we observed essentially all the DNA in the cytoplasm, even in cells expressing the encoded marker gene. Furthermore, we have observed the exclusion of fluorescent pDNA from the re-forming nuclei after mitosis, suggesting that mitosis itself fails to provide free access to the nuclear compartment. The strict sorting mechanism of bona fide nuclear components therefore inhibits the nuclear partitioning of non-chromatin molecules, such as exogenous pDNA, even if the pDNA had entered the nucleus prior to mitosis. We now show that association of a molecule with chromatin during mitosis, via a chromosome targeting signal (CTS) enhances nuclear localization of the compound in dividing cells.

In all higher eukaryotic cells the NE temporarily breaks down during mitosis enabling components normally confined to the cytoplasm to interact with components of the nucleus. At the end of anaphase NE-specific proteins accumulate in membrane patches that are in contact with the surface of the chromosomes. These patches expand and, during telophase at the end of mitosis, fuse along the surface of the chromosomes leaving very little free aqueous volume trapped inside (Chu A et al. 1998, Yang L et al. 1997, Cox L S et al. 1995, Georgatos S D et al. 1999). This reformation of the nuclear envelope effectively excludes most molecules which are not tightly associated with the chromatin or the newly forming nuclear envelope from being included within the newly formed nuclei. The result of this exclusion is a much decreased concentration of a compound of interest in the nuclei of daughter cells following cell division. By attaching the compound to a CTS, an increased percentage of the delivered compound (cargo compound) is retained in the nuclei of dividing cells. The cargo compound is attached to the CTS through either a covalent or non-covalent bond. Any method known in the art for covalently linking two compounds is considered to be within the scope of the invention provided the bond is sufficiently stable to persist inside a living eukaryotic cell and neither the bond itself nor forming the bond inactivates the CTS or the cargo compound. An exemplary non-covalent linkage that may be used with the invention comprises the well known streptavidin/avidin-biotin linkage. Stable antibody-antigen interactions may also be used to link a cargo compound with a CTS.

A Chromosome Targeting Signal (CTS) is defined in this specification as a molecule that enhances localization of an associated compound such as a nucleic acid, protein, drug or transfection reagent, to within the nucleus of a dividing eukaryotic cell through its interaction with chromatin or nuclear envelope during the latter stages of cell mitosis. Targeting of the compound to within the nucleus is not dependent on transport through a nuclear pore complex. The CTS can be a protein, peptide, protein fragment, antibody, antibody fragment, or a synthetic or natural molecule that interacts with mitotic chromosomes or other mitotic component such that the molecule is contained in the nucleus when the nuclear envelope reassembles at the end of mitosis.

An exemplary CTS is the Ki-67 protein, a component of mitotic chromosomes. The Ki-67 protein is a ˜395 kDa protein that shares structural similarities (forkhead-associated domain) with other proteins known to be involved in cell cycle regulation. Ki67 moves from within the nucleolus to the perichromosomal layer during mitosis. The binding pattern and timing of binding indicate that the Ki-67 protein, the KiF fragment and an anti-Ki-67 antibody monoclonal antibody are effective chromosome targeting signals.

Another exemplary CTS is the peptide comprising sequences derived from the human Herpes virus latent nuclear antigen 1 (LNA-1). The LNA-1 protein is responsible for the stable maintenance of Herpes virus by concomitantly binding to both the chromatin and the viral episomes, thus ensuring the transmission of the viral genome to the nuclei of the daughter cells after the host cell's division.

Other Chromosome Targeting Signals are readily identified using experiments similar to the examples provided in demonstrating the targeting ability of the Ki-67 protein, protein fragments, and anti-Ki-67 antibodies. Components of chromosomal structures present in or on chromatin either constitutively or during mitosis before the onset of telophase may be used as CTSs. Proteins that may serve a chromosomal targeting signal may be selected from the group comprising: structural proteins of the chromosomes or chromatin (histones), kinetochore proteins (CENP-B, CENP-C, CENP-D, CENP-E, CENP-F, CENP-G, CENP-H, INCENP, MCAK, ZW10), lamins, LAP2α, Emerin, Condensin, nuclear mitotic apparatus proteins (NuMa), ATRX, AKAP95, HA95, TTF-1, UBF (certain transcription factors known to remain strongly bound to chromosomes during mitosis), KLP38B, Rad17p, PNUTS (co-localizes with the chromosomes during telophase.), VP22, EBNA1, nucleolin, fibrillarin, B23, p52, p66, p103, perichromin, and peripherin. The CTS may be a purified naturally occurring protein, a recombinant protein, a protein fragment or peptide, or a synthetic mimetic of a functional CTS peptide. The CTS may also be a antibody or antibody fragment to a component of mitotic chromatin, such as the anti-Ki-67 antibody described herein.

Protein refers herein to a linear series of greater than 2 amino acid residues connected one to another via peptide bonds as in a polypeptide. A therapeutic effect of the protein in attenuating or preventing the disease state can be accomplished by the protein either staying within the cell, remaining attached to the cell in the membrane, or being secreted and dissociated from the cell where it can enter the general circulation and blood. Therapeutic proteins that stay within the cell (intracellular proteins) can be enzymes that clear a circulating toxic metabolite as in phenylketonuria. They can also cause a cancer cell to be less proliferative or cancerous (e.g., less metastatic), or interfere with the replication of a virus. Intracellular proteins can be part of the cytoskeleton (e.g., actin, dystrophin, myosins, sarcoglycans, dystroglycans) and thus have a therapeutic effect in cardiomyopathies and musculoskeletal diseases (e.g., Duchenne muscular dystrophy, limb-girdle disease). Other therapeutic proteins of particular interest to treating heart disease include polypeptides affecting cardiac contractility (e.g., calcium and sodium channels), inhibitors of restenosis (e.g., nitric oxide synthetase), angiogenic factors, and anti-angiogenic factors. A polymer is a molecule built up by repetitive bonding together of smaller units called monomers. Small polymer having 2 to about 80 monomers can be called oligomers. The polymer can be linear, branched network, star, comb, or ladder type. The polymer can be a homopolymer in which a single monomer is used or can be copolymer in which two or more monomers are used. Types of copolymers include alternating, random, block and graft.

The term polynucleotide, or nucleic acid or polynucleic acid, is a term of art that refers to a polymer containing at least two nucleotides. Nucleotides are the monomeric units of polynucleotide polymers. Polynucleotides with less than 120 monomeric units are often called oligonucleotides. Natural nucleic acids have a deoxyribose- or ribose-phosphate backbone. An artificial or synthetic polynucleotide is any polynucleotide that is polymerized in vitro or in a cell free system and contains the same or similar bases but may contain a backbone of a type other than the natural ribose-phosphate backbone. These backbones include: PNAs (peptide nucleic acids), phosphorothioates, phosphorodiamidates, morpholinos, and other variants of the phosphate backbone of native nucleic acids. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups such as, but not limited to, amines, alcohols, thiols, carboxylates, and alkylhalides. The term base encompasses any of the known base analogs of DNA and RNA. The term polynucleotide includes deoxyribonucleic acid (DNA) and ribonucleic acid (RNA) and combinations of DNA, RNA and other natural and synthetic nucleotides. The term includes plasmid DNA, single strand polynucleotide, double strand oligonucleotides, RNA, messenger RNA (mRNA), polynucleotides that induce RNA interference (siRNA, mRNA, etc), and anti-sense polynucleotides.

The process of delivering a nucleic acid to a cell has been commonly termed transfection or the process of transfecting and also it has been termed transformation. The term transfecting as used herein refers to the introduction of foreign nucleic acid or other biologically active compound into cells. The biologically active compound could be used to produce a change in a cell that can be therapeutic. The delivery of nucleic acid for therapeutic and research purposes is commonly called gene therapy. The delivery of nucleic acid can lead to modification of the genetic material present in the target cell.

A transfection reagent is a compound or compounds that bind(s) to or complex(es) with oligonucleotides and polynucleotides, and mediates their entry into cells. The transfection reagent also mediates the binding and internalization of oligonucleotides and polynucleotides into cells. Examples of transfection reagents include, but are not limited to, cationic lipids and liposomes, polyamines, calcium phosphate precipitates, histone proteins, polyethylenimine, and polylysine complexes. It has been shown that cationic proteins like histones and protamines, or synthetic cationic polymers like polylysine, polyarginine, polyornithine, DEAE dextran, polybrene, and polyethylenimine may be effective intracellular delivery agents, while small polycations like spermine are ineffective. Typically, the transfection reagent has a net positive charge that binds to the oligonucleotide's or polynucleotide's negative charge. The transfection reagent mediates binding of oligonucleotides and polynucleotides to cells via its positive charge (that binds to the cell membrane's negative charge) or via cell targeting signals that bind to receptors on or in the cell. For example, cationic liposomes or polylysine complexes have net positive charges that enable them to bind to DNA or RNA. Polyethylenimine, which facilitates gene transfer without additional treatments, probably disrupts endosomal function itself.

A molecule is modified, through a process called modification, by a second molecule if the two become bonded through a covalent bond. That is, the two molecules form a covalent bond between an atom form one molecule and an atom from the second molecule resulting in the formation of a new single molecule.

A chemical covalent bond is an interaction, bond, between two atoms in which there is a sharing of electron density.

Functional groups include cell targeting signals, nuclear localization signals, compounds that enhance release of contents from endosomes or other intracellular vesicles (releasing signals), and other compounds that alter the behavior or interactions of the compound or complex to which they are attached.

Cell targeting signals are any signals that enhance the association of the biologically active compound with a cell. These signals can modify a biologically active compound such as drug or nucleic acid and can direct it to a cell location (such as tissue) or location in a cell (such as the nucleus) either in culture or in a whole organism. The signal may increase binding of the compound to the cell surface and/or its association with an intracellular compartment. By modifying the cellular or tissue location of the foreign gene, the function of the biologically active compound can be enhanced. The cell targeting signal can be, but is not limited to, a protein, peptide, lipid, steroid, sugar, carbohydrate, (non-expressing) polynucleic acid or synthetic compound. Cell targeting signals such as ligands enhance cellular binding to receptors. A variety of ligands have been used to target drugs and genes to cells and to specific cellular receptors. The ligand may seek a target within the cell membrane, on the cell membrane or near a cell. Binding of ligands to receptors typically initiates endocytosis. Ligands include agents that target to the asialoglycoprotein receptor by using asialoglycoproteins or galactose residues. Other proteins such as insulin, EGF, or transferrin can be used for targeting. Peptides that include the RGD sequence can be used to target many cells. Chemical groups that react with thiol, sulfhydryl, or disulfide groups on cells can also be used to target many types of cells. Folate and other vitamins can also be used for targeting. Other targeting groups include molecules that interact with membranes such as lipids, fatty acids, cholesterol, dansyl compounds, and amphotericin derivatives. In addition viral proteins could be used to bind cells.

After interaction of a compound or complex with the cell, other targeting groups can be used to increase the delivery of the biologically active compound to certain parts of the cell.

Nuclear localizing signals enhance the targeting of the pharmaceutical into proximity of the nucleus and/or its entry into the nucleus through the nuclear pore during interphase of the cell cycle. Such nuclear transport signals can be a protein or a peptide such as the SV40 large T antigen NLS or the nucleoplasmin NLS. These nuclear localizing signals interact with a variety of nuclear transport factors such as the NLS receptor (karyopherin alpha) which then interacts with karyopherin beta. Several peptides have been derived from the SV40 T antigen. Other NLS peptides have been derived from the hnRNP A1 protein, nucleoplasmin, c-myc, etc.

Many biologically active compounds, in particular large and/or charged compounds, are incapable of crossing biological membranes. In order for these compounds to enter cells, the cells must either take them up by endocytosis, i.e., into endosomes, or there must be a disruption of the cellular membrane to allow the compound to cross. In the case of endosomal entry, the endosomal membrane must be disrupted to allow for movement out of the endosome and into the cytoplasm. Either entry pathway into the cell requires a disruption of the cellular membrane. Compounds that disrupt membranes or promote membrane fusion are called membrane active compounds. These membrane active compounds, or releasing signals, enhance release of endocytosed material from intracellular compartments such as endosomes (early and late), lysosomes, phagosomes, vesicle, endoplasmic reticulum, Golgi apparatus, trans Golgi network (TGN), and sarcoplasmic reticulum. Release includes movement out of an intracellular compartment into the cytoplasm or into an organelle such as the nucleus. Releasing signals include chemicals such as chloroquine, bafilomycin or Brefeldin A1 and the ER-retaining signal (KDEL motif), viral components such as influenza virus hemagglutinin subunit HA-2 peptides and other types of amphipathic peptides. The control of when and where the membrane active compound is active is crucial to effective transport. If the membrane active agent is operative in a certain time and place it would facilitate the transport of the biologically active compound across the biological membrane. If the membrane active compound is too active or active at the wrong time, then no transport occurs or transport is associated with cell rupture and cell death. Nature has evolved various strategies to allow for membrane transport of biologically active compounds including membrane fusion and the use of membrane active compounds whose activity is modulated such that activity assists transport without toxicity. Many lipid-based transport formulations rely on membrane fusion and some membrane active peptides' activities are modulated by pH. In particular, viral coat proteins are often pH-sensitive, inactive at neutral or basic pH and active under the acidic conditions found in the endosome.

Another functional group comprises compounds, such as polyethylene glycol, that decrease interactions between molecules and themselves and with other molecules. Such groups are useful in limiting interactions such as between serum factors and the molecule or complex to be delivered.

Spacer group—Preferred spacer groups include, but are not limited to C1-C12 alkyl, C1-C12 alkenyl, C1-C12 alkynyl, C6-C18 aralkyl, C6-C18 aralkenyl, C6-C18 aralkynyl, ester, ether, ketone, alcohol, polyol, amide, amine, polyglycol, polyether, polyamine, thiol, thio ether, thioester, phosphorous containing, and heterocyclic.

### EXAMPLES

### Example 1

### Exogenous DNA and Large Molecules are Excluded from the Nucleus Following Mitosis

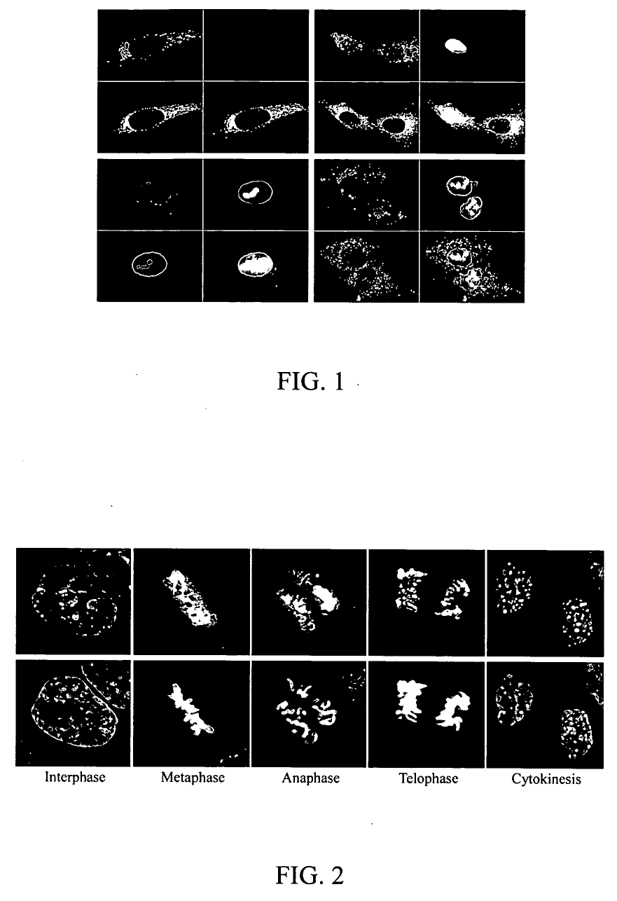

In order to visualize the amount of pDNA in the nucleus and in the cytoplasm after mitosis, we injected a mixture of unlabeled pEYFP-Nuc plasmid, a fluorescently labeled pDNA and fluorescent dextran into HeLa cells. Following either cytoplasmic or nuclear microinjections, the physical location of the labeled DNA and dextran was detected both before and after mitosis. FIG. 1 demonstrates that in cells that had gone through mitosis both the dextran and the DNA became efficiently excluded from the newly formed nuclei. It was striking that both dextran and pDNA were excluded from the re-forming nuclei extremely efficiently, even if they had been injected into the nucleus. We hypothesize that, lacking a targeting mechanism to accumulate in the vicinity of the chromosomes, the fraction of delivered compound packaged into the newly formed nucleus is proportional to the volume of cytoplasm entrapped within the re-forming NE on the surface of telophase chromosomes. This observation fits well the estimation that<1% of the cytoplasmically delivered DNA reaches the nucleus, or remains in the nucleus, after mitosis.

### Example 2

### Expression of Microinjected DNA

We found that pDNA expressed several hundred-fold more efficiently when microinjected into the nucleus rather than into the cytoplasm. Table 1 shows the results in terms of the number of pEYFP-Nuc molecules injected per cell. In order to enable 50% of the cells to express EYFP-Nuc, it required injection of approximately 2000 copies into the cytoplasm. Conversely, injection of only 3 copies into the nucleus yielded 50% expression: a 700-fold difference. HeLa cells were injected with the indicated amount of pEYFP-Nuc plus 50 ng/μl inert carrier DNA to prevent loss of DNA from adsorption. The injection volume was 0.42 μl for cytoplasmic injection and 0.15 pl for nuclear injection. This volume corresponds to approximately 10% of the compartment volume. EYFP expression was assayed 20 hours after injection by fluorescent microscopy.

TABLE 1
Effect of pDNA concentration on expression levels in
non-synchronized HeLa cells. Cells were not scored for
mitosis; during the 20 hours incubation time approximately
70-80% of the cell population divided.

| | pEYFP molecules injected/cell | | % cells expressing YFP | |
|---|---|---|---|---|
| pEYFP-Nuc | cytoplasmic | nuclear | cytoplasmic | nuclear |
| (ng/μl) | injection | injection | injection | injection |
| 0.02 | 1.6 | 0.6 | 0.0 | 10.3 |
| 0.1 | 8 | 2.9 | 0.6 | 46.9 |
| 1 | 80 | 29 | 5.4 | 74 |
| 2 | 160 | 57 | 31 | 95 |
| 10 | 800 | 286 | 31 | 96 |
| 20 | 1600 | 571 | 41 | 100 |
| 25 | 2000 | 714 | 53 | 100 |

### Example 3

### Effect of Mitosis of Expression of Microinjected pDNA

We also evaluated individual injected cells, both for cell division and for marker gene expression. The data show that cells into which pDNA was injected cytoplasmically were able to express GFP without going through mitosis. Therefore some small fraction of the injected pDNA is able to enter the intact interphase nucleus. However, expression increased in cells that had gone through mitosis: from 28% to 50% after cytoplasmically injecting 10 ng/μl pEYFG-Nuc and from 50% to 90% after cytoplasmically injecting 1,000 ng/μl pEYFP-Nuc. However, expression levels never attained 100% in dividing cells, even when cytoplasmically injected with 1,000 ng/μl or 8×10⁴ copies of pEYFP-Nuc. Conversely, for nuclear injection of pDNA, a few hundred copies per nucleus results in 100% expression. Based on these observations we conclude that the amount of DNA that can enter the nucleus during mitosis is more than the amount entering through NPCs during interphase. However, even during mitosis, the amount of cytoplasmic DNA that gains access to the nucleus is less than 1%.

### Example 4

### Limitations of Enhancement of Gene Expression Using NLS Peptides

We have also experimented with promoting nuclear DNA uptake by the stable attachment of multiple copies of NLS peptides to linear DNA. We used a linear, <1 kb minimal expression cassette with a single biotin on one end. Streptavidin was covalently conjugated to either a 39 residue peptide (H-CKKKSSSDDEATADSQHSTPPKKKRK-VEDPKDFPSELLS; SEQ ID 1) containing the wild type SV40 NLS (Yoneda Y et al. 1992), or to a mutant version known to be transport deficient. Judged by SDS-PAGE the number of peptides per SA monomer was estimated to be 2. The conjugates were added to a linear, end-biotinylated and fluorescently labeled DNA, followed by microinjection into the cytoplasm of HeLa cells. Complexes with the functional NLS expressed the GFP marker gene 7 times more efficiently than complexes with the mutant NLS (an increase from 1.5% to 10.9%). Thus, using a stable bond between the linear DNA and multiple copies of a strong NLS, a 7-fold increase in expression efficiency could be obtained (Ludtke J J et al. 1999). However, the data also show that NLS-mediated uptake of DNA is size dependent, with nuclear targeting efficiency dropping dramatically for DNA molecules larger than 1 kb.

### Example 5

### Localization of Ki-67 Protein to Mitotic Chromosomes

HeLa cell cultures were enriched in mitotic cells by a double thymidine block. 9-10 h after releasing the cells from the block they were fixed with 4% formaldehyde and permeabilized with TritonX-100. An in vitro binding assay was performed with monoclonal antibodies (MAbs) against histone H1 (StressGene), Nup62, topoisomeraseII, mitosin, and Ki-67 (Transduction Laboratories). The antibodies were detected with an Alexa488-labeled anti-mouse IgG (Alexa488-anti-MIgG) secondary antibody.

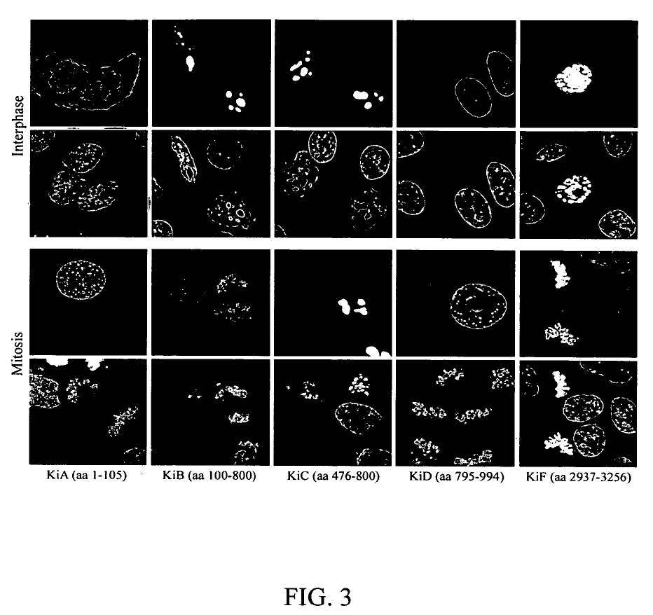

The anti-Histone H1 MAb gave weak, finely punctate nuclear staining during interphase, and chromosomal staining during mitosis. The ends of the chromosomes stained more intensely than the centromeric regions. During interphase Nup62 localized to a rim around the nucleus, with some additional weak, diffuse staining in the cytoplasm and nucleoplasm. During mitosis it was initially evenly dispersed throughout the cytoplasm, while fully excluded from the chromosomal volume. After anaphase, Nup62 started to accumulate on the outside surface of the chromosome cluster, and by the end of cytokinesis, it again localized to a rim around the new nucleus. The anti-mitosin MAb yielded non-nucleolar staining in the interphase nucleus. During mitosis most of mitosin was evenly dispersed in the cytoplasm, and a fraction of the antigen formed bright, small spots at the kinetochore of each chromosome. The interphase staining pattern of TopoisomeraseIIβ was very similar to Histone H1. During mitosis, TopoIIβ was barely detectable, suggesting that the epitope to which the MAb binds was not accessible in the condensed mitotic chromosome. The anti-Ki-67 antibody showed intense peri-nucleolar staining during interphase, and re-distributed to around the chromosomes during metaphase and anaphase. As shown in FIG. 2, the signal was strong and localized exclusively to the peri-chromosomal sheath. There was no detectable fluorescence in the cytoplasm. The diffuse peri-chromosomal staining became more distinctly co-localizing with chromosomes by telophase. After cytokinesis the antigen disengaged from the chromosomes and migrated back to nucleoli.

### Example 6

### Localization of Anti-Ki-67 Monoclonal Antibodies to Mitotic Chromosomes

MAbs were diluted to 25 ng/l in intracellular buffer (10 mM PIPES pH 7.2, 140 mM KCl, 1 mM MgCl₂) and injected into the cytoplasm of HeLa cells. The cells were processed for microscopy 3-4 h and 20-24 h after injection. The location of the injected MAb was determined by staining with Alexa488-anti-mouse IgG.

The staining pattern of the microinjected anti-Ki-67 MAb was similar to that observed the for Ki-67 protein as described above. The MAb was strongly anchored to the chromosomes of mitotic cells with no detectable antigen left in the cytoplasm during mitosis. This result indicates that the interaction between the anti-Ki-67 antibody and the Ki-67 protein is sufficiently strong to permit use of the anti-Ki-67 antibody as a CTS. Surprisingly, nuclear entry of the MAb did not require mitosis, suggesting that the anti-Ki-67 MAb is actively transported along with the Ki-67 protein into the nucleus through NPCs. This MAb may therefore be used, not only as a CTS, but also as an NLS, enhancing nuclear localization of attached cargo/compounds during both interphase and mitosis.

The antibody had not apparent toxic effects at the 25 ng/μl concentration, ˜5×10⁴ IgG molecules per cell, used. Based on morphology the cells looked healthy and were dividing. Therefore, interference with normal cellular functions is unlikely for these MAbs, at this concentration.

### Example 7

### Mapping the Chromosome Targeting Domain of Ki-67

The primary sequence of the Ki-67 protein has been determined (Schluter C et al. 1993), and its domain structure has been partially characterized (Schluter C et al. 1993, Endl E et al. 2000, MacCallum D E et al. 2000, Sueishi M et al. 2000, Scholzen T et al. 2002). None of the previous studies identified the domain responsible for directing Ki-67 to the peri-chromosomal sheath during mitosis. We made a series of EYFP-fusion constructs using various fragments of Ki-67. The fragments covered amino acid residues 1-105 (KiA), 100-800 (KiB), 476-800 (KiC), 795-994 (KiD), and 2937-3256 (KiF). The largest domain of the protein (KiE, amino acids 995-2936) contains 16 repeats of a 120 amino acid motif. The subcellular distribution of a small fragment of this domain, which contains the 6th repeat motif (residues 1604-1725), with some flanking sequences was analyzed. This fragment did not accumulate in the nucleus during interphase, and did not bind to mitotic chromosomes (data not shown). The characteristic staining pattern of the other five domains in transiently transfected HeLa cells is shown in FIG. 3. The N-terminal KiA domain, also called the forkhead associated domain (Sueishi Met al. 2000), partially localized to the nucleus in interphase cells, while some peptide remained in the cytoplasm. During mitosis KiA showed diffuse cytoplasmic staining with scattered, bright spots (FIG. 3. KiA panels). KiB contains the protein's nucleolar localization signal. Both the full length KiB and its C-terminal half, KiC, accumulated in nucleoli in interphase cells. During mitosis, they became evenly dispersed throughout the cytoplasm, with a weak peri-chromosomal accumulation visible in some cells (FIG. 3. KiB and KiC panels). The small domain between the nucleolar targeting domain and the 16 repeat domains, KiD accumulated in the nuclei very efficiently, but it was excluded from nucleoli. During mitosis KiD localization was similar to KiA (FIG. 3. KiD panels). The C-terminal KiF fragment, which had been shown to bind both DNA and the HP1 protein (MacCallum D E et al. 2000, Scholzen T et al. 2002), co-localized with chromosomes during mitosis (FIG. 3. KiF panels). KiF, containing the C-terminal 320 residues, is therefore sufficient for targeting the peri-chromosomal protein layer during mitosis. Targeting with this fragment was just as efficient as targeting of the full-length protein (FIG. 2). Thus, the KiF protein fragment is a functional CTS, capable of targeting an attached fluorescent protein to mitotic chromosomes and into the newly formed nuclei of daughter cells.

### Example 8

### Using a Synthetic Peptide as a CTS Compound to Target a Protein Cargo Molecule to the Nucleus During Mitosis

A peptide representing amino acid residues 5-22 of the latent nuclear antigen (LNA-1) of human Herpesvirus 8 (HHV-8) (Piolot T et al. 2001) was synthesized using standard peptide synthesis procedures. The peptide contained an N-terminal biotin moiety linked to the first glycine residue by a PEG₁₁ spacer: Biotin-PEG₁₁-G₅MRLRSGRSTGAPLTRGS₂₂ (Swiss Protein Database/TrEMBL Accession number Q9QR71; SEQ ID 2, M. W. 2,687 dalton). The peptide was complexed with Cy3-labeled streptavidin (Cy3-SA) at a 4-fold molar excess of biotin over biotin binding sites in isotonic (5%) glucose solution buffered with 20 mM MOPS, ph 7.5. The Cy3-SA was prepared using the Mono-Reactive Cy3-NHS-Ester Protein Labeling Kit (Amersham) and was purified by gel filtration. The complex was incubated at room temperature for 30 minutes followed by a 5-minute spin at 14,000 g. The clear supernatant was used for microinjecting HeLa cells (human cervical carcinoma cell line) either into the cytoplasm or into the nucleus, as described in Ludtke J J et al. (2002). The cell cultures were fixed with 4% formaldehyde in PBS (FA/PBS) 13 hours later and were processed for microscopy by counter-staining with 13 nM TO-PRO®-3 DNA stain and 16.5 nM Alexa488-Phalloidin actin stain (Invitrogen). Fluorescent images were collected using an LSM510 confocal laser scanning microscope (Carl Zeiss, Thornwood, N.Y.).

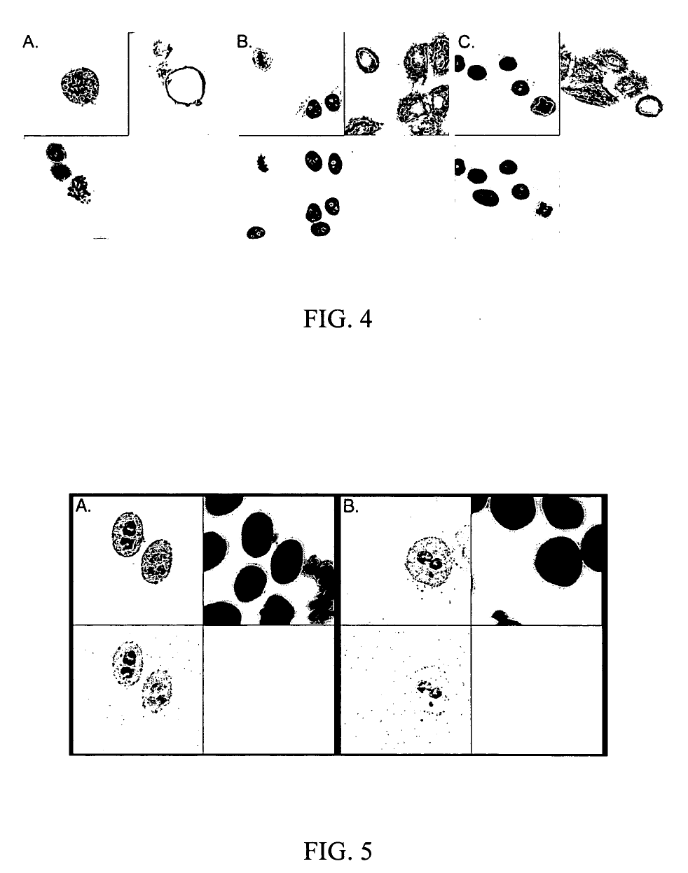

Results: Microscopic images of the injected HeLa cells revealed Cy3-SA cargo molecules in the nuclear compartment of interphase cells both after cytoplasmic and nuclear microinjections (FIG. 4). Therefore, the complex likely entered the interphase nucleus after cytoplasmic injections by active transport mechanism through the nuclear pore. Since the LNA-1 peptide does not contain a known nuclear localization signal (NLS), it is likely that nuclear transport is mediated through interaction of the peptide with its target protein on chromatin. While the complex was evenly distributed in the nucleoplasm during interphase, it efficiently accumulated on the surface of condensed mitotic chromosomes during all phases of mitosis (FIG. 4). Chromosomal association persisted during the assembly of the nuclear envelope. Therefore, the LNA-1 peptide functioned as a CTS.

### Example 9

### Anti-Ki-67 Antibodies are Effective CTS

A set of mouse hybridoma clones was generated by the genetic immunization of ICR mice followed by the standard protocol for hybridoma production. The expression vector pCI-Ki67 encoded residues 1547-1742 of the human Ki-67 antigen under control of the human cytomegalovirus (CMV) promoter in the pCI vector (Promega Corporation, Madison, Wis.). The naked plasmid DNA was diluted in Ringers' solution and a volume equal to 10% of the animals' body weight was injected into the tail vein of mice in 6 to 7 seconds according to published hydrodynamic gene delivery protocols. The injections were repeated 2, 3, 4 and 5 weeks after the initial injection. Splenocytes from candidate mice were harvested four days after a final pDNA boost given 104 days after the initial pDNA injection. Hybridoma clones were initially screened for specific antibody production using ELISA, followed by screening the positive supernatants by immunocytochemistry to select antibodies that bound to mitotic chromosomes. For the immunocytochemistry HeLa cells were grown on glass cover slips, fixed in 4% formaldehyde in PBS, permeabilized with 0.2% Triton-X-100 in PBS and blocked with 2 mg/ml BSA in PBS. Culture supernatants from hybridoma clones were diluted 1:5, applied to the slides and incubated 1 hour at RT. The secondary antibody was Cy3-labeled anti-Mouse IgG (H+ L) F(ab′)2 fragment (Jackson ImmunoResearch Laboratories, West Grove, Pa.), diluted 1:600 in 2 mg/ml BSA in PBS. The samples were counter-stained with 13 nM TO-PRO®-3 DNA stain and 16.5 nM Alexa488-Phalloidin actin stain. Fluorescent images were collected using a Zeiss LSM510 confocal laser scanning microscope.

Based on ELISA, immunocytochemistry and Western blotting results >40 hybridoma cell lines were identified that produced MAbs reactive with various epitopes of the human Ki-67 antigen. Antibodies that did not bind to the hyperphosphorylated form of the Ki-67 antigen on the surface of the mitotic chromosomes were eliminated as CTS compounds. One of the MAbs that gave intense staining during all phases of the cell cycle was identified. This hybridoma cell line was cloned twice and expanded for large-scale production of IgG.

The purified Ki-67 reactive IgG molecules were chemically conjugated to biotin using EZ-Link Sulfo-NHS-LC-LC-Biotin (Pierce) at 30-fold molar excess. This labeling reagent provides a long, (30.5 Angstrom), spacer arm to prevent steric hindrance between the CTS IgG and its cargo molecule. The excess reagent was removed by gel filtration and the biotinylated immunoglobulins (Biot-IgG) were complexed with Oregon-Green488-Streptavidin (OG-SA; Molecular Probes). An excess of biotins over biotin binding sites was used to reduce formation of cross-linked, multi-molecular complexes. The sample was incubated at room temperature for 30 minutes followed by a 5-minute spin at 14,000 g to remove any large aggregates. The clear supernatant containing Biot-IgG-OG-SA complexes was microinjected into the cytoplasm of HeLa cells. The cells were fixed and stained with 13 nM TO-PRO®-3 DNA stain 16 hours later. The location of the IgG was detected by a Cy3-labeled anti-Mouse IgG (H+L) F(ab′)2 fragment (Jackson ImmunoResearch Laboratories, West Grove, Pa.), diluted 1:600 in 2 mg/ml BSA in PBS.

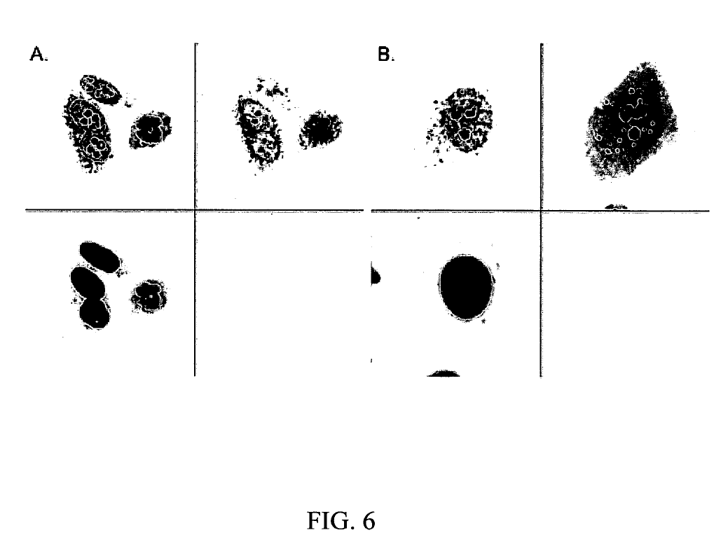

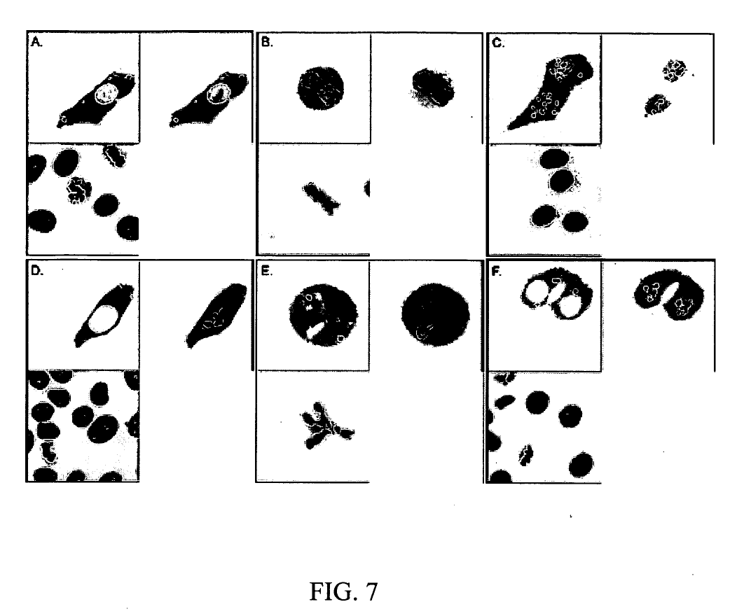

Results: During the 16 hours of incubation time between microinjection and fixation of most of the injected cells went through mitosis and generated two daughter cells. FIG. 5 shows the subcellular location of the CTS compound IgG and its cargo, the OG-SA, both in divided and undivided cells (FIG. 5). In both cases the two molecules co-localize perfectly and are targeted to the perinucleolar rim within the nucleus, typical of the location for Ki-67 during interphase. This suggests that the complexes remained stably attached within the cell and were able to enter the nuclear compartment together both during interphase and during mitosis. Transport through the nuclear pore in interphase cells likely occurs through binding to the target Ki-67 molecule in the cytoplasm. No OG-SA signal was observed apart from Ki67, indicating binding of the antibody to Ki67 throughout the experiment and cell division. Thus, the anti-Ki-67 antibody functionally targeted streptavidin protein to the nucleus.

### Example 10

### Conjugation of the Ki-67-Reactive IgG to Streptavidin in Order to Facilitate the Nuclear Targeting of Various Biotinylated Compounds

Anti-Ki67 IgG was treated with a 45-fold molar excess of 2-Iminothiolane-HCl (Traut's reagent; Pierce) in PBS to introduce sulfhydryl groups, followed by purification on a gel-filtration column. Streptavidin was maleimide-activated by incubating it with a 20-fold molar excess of Sulfo-SMCC (Pierce) in 0.1 M NaPO₄ buffer pH8.0 and was also purified by gel-filtration. Immediately after gel-filtration, the thiolated-IgG and the maleimide-SA were combined in 0.1M NaPO₄ buffer pH7.0 at a 2:1 weight ratio and were incubated at room temperature for 1 hour, followed by an overnight incubation at 4° C. Fluorescein-Biotin (Pierce) was used as a small-molecule cargo to test the ability of the IgG-SA conjugate to target the cargo to mitotic chromosomes and the nuclear compartment.

Fluorescein-Biotin was complexed with the conjugate at a 5× molar excess over estimated biotin-binding sites, and the excess was removed by gel-filtration after 15 minutes of incubation. Bovine serum albumin (BSA) was Cy3 labeled and biotinylated to serve as a protein cargo molecule. Cy3-labeling was performed as described above for SA. The purified Cy3-BSA was then biotinylated. In a similar way, a 70 kDa amine-dextran polymer was double-labeled with Cy3 and biotin to serve as another cargo substance. Both the Cy3-Biot-BSA and Cy3-Biot-Dex₇₀ were complexed with the IgG-SA conjugate at an estimated equimolar ratio in PBS. Multi-molecular complexes and large aggregates were removed by spinning the samples at 14,000 g for 5 minutes. The clear supernatants were microinjected into the cytoplasm of HeLa cells. The cells were fixed and counter-stained with 13 nM TO-PRO®-3 DNA stain 16 hours later. The location of the IgG was detected either by a Cy3-labeled anti-Mouse IgG (H+ L) F(ab′)2 fragment (Jackson ImmunoResearch Laboratories), or by an Alexa Fluor 488-labeled polyclonal goat anti-Mouse IgG (H+L) secondary antibody. Images were collected by confocal microscopy as in Example 1.

Results: Anti-Ki67 IgG-SA conjugate complexed with either Fluorescein-Biotin or Cy3-Biotin-BSA accumulated cargo molecules to nuclei during interphase and during mitosis. FIG. 6 shows that the cargoes were targeted to the characteristic sub-cellular location of Ki-67: perinucleolar interphase nucleus and the surface of the condensed chromosomes during mitosis. Cy3-Biot-Dex₇₀ remained excluded from interphase nuclei unless the cell had gone through mitosis. FIG. 7A shows an undivided HeLa cell. FIG. 7B shows a metaphase cell, in which the CTS-dextran cargo complex accumulated on the surface of chromosomes. FIG. 7C shows that, at the end of mitosis, the cargo was targeted to the chromatin during mitosis and to the nuclear compartment in both daughter cells. Cytoplasmic staining of the dextran cargo is likely the result of the high concentration of CTS-dextran conjugates injected, saturating the number of available Ki-67 binding sites. Panels D-E-F of FIG. 7 show the distribution of unconjugated anti-Ki-67 IgG and SA complexed with the Cy3-Biot-Dex₇₀. FIG. 7E-F show that Cy3-Biot-Dex₇₀ complexed with unconjugated SA did not accumulate around chromatin during mitosis and was therefore efficiently excluded from the newly formed nuclei of the daughter cells after mitosis.

The foregoing is considered as illustrative only of the principles of the invention. Furthermore, since numerous modifications and changes will readily occur to those skilled in the art, it is not desired to limit the invention to the exact construction and operation shown and described. Therefore, all suitable modifications and equivalents fall within the scope of the invention.

We claim:

1. A process for enhancing nuclear localization of a cargo compound comprising:
forming a composition comprising the cargo compound linked to a Ki67 chromosome targeting signal and delivering the composition to a dividing cell.

2. The process of claim 1 further comprising associating the composition with a transfection agent.

3. The process of claim 1 wherein the chromosome targeting signal comprises a peptide.

4. The process of claim 3 wherein the peptide is selected from the group consisting of: protein, protein fragment, synthetic protein, synthetic peptide, and recombinant proteins.

5. The process of claim 4 wherein the peptide is selected from the group consisting of: anti-Ki67 protein antibody and anti-Ki67 protein antibody fragment.

6. The process of claim 1 wherein the cargo compound is linked to the chromosomal targeting signal via a non-covalent interaction.

7. The process of claim 6 wherein the non-covalent interaction consists of a streptavidin-biotin interaction.

8. The process of claim 1 wherein the cargo compound is linked to the chromosomal targeting signal via a covalent bond.

9. The process of claim 8 wherein covalent bond consists of a disulfide bond.

10. The process of claim 1 wherein the cell is an tumor cell.

11. The process of claim 10 wherein delivery of the compound results in cell death.

12. The process of claim 1 wherein the cargo compound comprises a small molecule drug.

13. The process of claim 1 wherein the cargo compound comprises a protein.

14. A process for enhancing nuclear localization of a cargo compound comprising:
forming a composition comprising the cargo compound linked to a human herpesvirus 8 latent nuclear antigen-1 (LNA-1) chromosome targeting signal and delivering the composition to a dividing cell.

15. The process of claim 14 wherein the chromosome targeting signal comprises a peptide.

16. The process of claim 15 wherein the peptide is selected from the group consisting of: anti-LNA-1 antibody and anti-LNA-1 antibody fragment.

17. The process of claim 14 wherein the cargo compound is linked to the chromosomal targeting signal via a non-covalent interaction.

18. The process of claim 14 wherein the cargo compound is linked to the chromosomal targeting signal via a covalent bond.

19. The process of claim 14 wherein the cargo compound comprises a small molecule drug.

20. The process of claim 14 wherein the cargo compound comprises a protein.


SEQUENCE LISTING (2 sequences)

SEQ ID NO: 1 - protein, 39 aa, Simian virus 40
CKKKSSSDDEATADSQHSTPPKKKRKVEDPKDFPSELLS

SEQ ID NO: 2 - protein, 18 aa, Human Herpesvirus 8
GMRLRSGRSTGAPLTRGS